(19) United States
(12) Patent Application Publication
(10) Pub. No.: US 2021/0298619 A1
(43) Pub. Date: Sep. 30, 2021

(54) BIOMETRIC SENSOR HEAD ASSEMBLY

(71) Applicant: VALENCELL, INC., Raleigh, NC (US)

(72) Inventors: Shawn M. STEPHENSON, Raleigh, NC (US); Steven Francis LeBOEUF, Raleigh, NC (US)

(21) Appl. No.: 17/057,931

(22) PCT Filed: May 29, 2019
(86) PCT No.: PCT/US2019/034386

§ 371 Date: Nov. 23, 2020

Related U.S. Application Data
(60) Provisional application No. 62/678,433, filed on May 31, 2018.

(51) Int. Cl. A61B 5/024; A61B 5/00; H04R 25/00

(52) U.S. Cl. CPC A61B 5/02416; A61B 5/6817; A61B 2562/166; H04R 25/609; H04R 25/604

(57) ABSTRACT

A sensor head assembly includes a body member having an internal passage. An optical source is secured to the body member at a first location, and an optical detector is secured to the body member at a second location. An audio driver is secured to the body member. An ear gel configured to be inserted within an ear canal is attached to the body member and has at least a portion thereof transparent to one or more wavelengths of light. A first light guide is secured to an inner surface of the ear gel via a respective first end, and a second light guide is secured to the inner surface of the ear gel via a respective first end. A second end of the first light guide is in optical communication with the optical source, a second end of the second light guide is in optical communication with the optical detector.

### RELATED APPLICATION

This application claims the benefit of and priority to U.S. Provisional Patent Application No. 62/678,433 filed May 31, 2018, the disclosure of which is incorporated herein by reference as if set forth in its entirety.

### FIELD OF THE INVENTION

The present invention relates generally to monitoring devices and, more particularly, to optical sensor devices.

### BACKGROUND OF THE INVENTION

There is growing market demand for personal health and environmental monitors, for example, for gauging overall health and metabolism during exercise, athletic training, dieting, daily life activities, sickness, and physical therapy. However, traditional health monitors and environmental monitors may be bulky, rigid, and uncomfortable—generally not suitable for use during daily physical activity. Moreover, wearable fitness trackers have mostly been focused on wrist-worn form-factors that are used for sports and fitness applications rather than form-factors routinely used by those having health conditions.

Sensors for detecting biometric signals, such as vital signs and other physiological information, are configured to isolate the biometric signals from other spurious signals and deliver biometric readings, such as heart rate, respiration rate, blood pressure, etc., to the user. Unfortunately, spurious signals that may be difficult to isolate from a biometric signal are associated with physical movement (e.g., physical exercise, such as walking, running, daily activities, etc.) of a sensor relative to the user or the environment of the user (e.g., sunlight, room light, humidity, ambient acoustical or electromagnetic noise, temperature extremes or changes in temperature, etc.).

Previous ways of isolating heart rate signals from other signals include the use of passive and active signal processing algorithms, increasing optical sensor output and displacing the optical source from the photodetector, and pushing the sensor more firmly against the user so as to limit the effects of physical movement on the heart rate signal.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, a sensor head assembly includes a body member sized to fit within an ear canal of a subject. The body member may have a generally cylindrical configuration, but other shapes may be utilized. The body member includes an inlet port and an exit port at respective opposite ends of the body member, and an internal acoustic passage extends between the inlet port and the exit port. In some embodiments, the acoustic passage extends along a longitudinal centerline of the body member. In other embodiments, at least a portion of the acoustic passage extends along a non-centerline portion of the body member.

An optical source is secured to the body member at a first location, and an optical detector is secured to the body member at a second location different from the first location. The optical source and optical detector are associated with a sensor, such as a photoplethysmography (PPG) sensor, for monitoring/detecting physiological information from a person wearing the sensor head assembly. In some embodiments, the first and second locations are circumferentially spaced apart from each other relative to the body member, and in other embodiments, the first and second locations are adjacent each other. An audio driver is secured to the body member and is in acoustic communication with the acoustic passage of the body member. In some embodiments, the audio driver has a sound outlet tube, and the sound outlet tube is secured within the acoustic passage of the body member via the inlet port. In some embodiments, at least a portion of the audio driver, optical source and optical detector are encapsulated within a hydrophobic material.

An ear gel is attached to the body member, and includes opposite inner and outer surfaces. In some embodiments, the ear gel is configured to be removably secured to the body member. The ear gel is configured to be inserted within the ear canal of the subject and is transparent to one or more selected wavelengths of light, or has one or more portions that are transparent to one or more selected wavelengths of light. A first light guide having opposite first and second ends is secured to the inner surface of the ear gel via the first end of the first light guide, and a second light guide having opposite first and second ends is secured to the inner surface of the ear gel via the first end of the second light guide. The second end of the first light guide is in optical communication with the optical source and the first light guide is configured to deliver light from the optical source into a first region of the ear canal. The second end of the second light guide is in optical communication with the optical detector the second light guide is configured to collect light from a second region of the ear canal and deliver collected light to the optical detector.

According to other embodiments of the present invention, a sensor head assembly includes a body member sized to fit within an ear canal of a subject. The body member may have a generally cylindrical configuration, but other shapes may be utilized. The body member includes an inlet port and an exit port at respective opposite ends of the body member, and an internal acoustic passage extends between the inlet port and the exit port. In some embodiments, the acoustic passage extends along a longitudinal centerline of the body member. In other embodiments, at least a portion of the acoustic passage extends along a non-centerline portion of the body member. An audio driver is secured to the body member and is in acoustic communication with the acoustic passage of the body member. In some embodiments, the audio driver includes a sound outlet tube that is secured within the acoustic passage of the body member via the inlet port.

An annular member surrounds at least a portion of the audio driver. An optical source is secured to the annular member at a first location and an optical detector secured to the annular member at a second location different from the first location. The optical source and optical detector are associated with a sensor, such as a PPG sensor, for monitoring/detecting physiological information from a person wearing the sensor head assembly. In some embodiments, the first and second locations are circumferentially spaced apart from each other relative to the annular member, and in other embodiments, the first and second locations are adjacent each other. In some embodiments, at least a portion of the audio driver, annular member, optical source and optical detector are encapsulated within a hydrophobic material.

An ear gel is attached to the body member, and includes opposite inner and outer surfaces. In some embodiments, the ear gel is configured to be removably secured to the body member. The ear gel is configured to be inserted within the ear canal of the subject and is transparent to one or more selected wavelengths of light or has one or more portions that are transparent to one or more selected wavelengths of light. A first light guide having opposite first and second ends is secured to the inner surface of the ear gel via the first end of the first light guide, and a second light guide having opposite first and second ends is secured to the inner surface of the ear gel via the first end of the second light guide. The second end of the first light guide is in optical communication with the optical source and the first light guide is configured to deliver light from the optical source into a first region of the ear canal. The second end of the second light guide is in optical communication with the optical detector the second light guide is configured to collect light from a second region of the ear canal and deliver collected light to the optical detector.

According to other embodiments of the present invention, a sensor head assembly includes a body member sized to fit within an ear canal of a subject. The body member may have a generally cylindrical configuration, but other shapes may be utilized. The body member includes an inlet port and an exit port at respective opposite ends of the body member, and an internal acoustic passage extends between the inlet port and the exit port. In some embodiments, the acoustic passage extends along a longitudinal centerline of the body member. In other embodiments, at least a portion of the acoustic passage extends along a non-centerline portion of the body member. An audio driver is secured to the body member and is in acoustic communication with the acoustic passage of the body member. In some embodiments, the audio driver includes a sound outlet tube that is secured within the acoustic passage of the body member via the inlet port.

A flexible printed circuit is secured to the audio driver. In some embodiments the flexible printed circuit may surround or at least partially surround an outer surface of the audio driver. An optical source is secured to the flexible printed circuit at a first location on the flexible printed circuit and an optical detector secured to the flexible printed circuit at a second location on the flexible printed circuit different from the first location. The optical source and optical detector are associated with a sensor, such as a PPG sensor, for monitoring/detecting physiological information from a person wearing the sensor head assembly. In some embodiments, the first and second locations are circumferentially spaced apart from each other relative to the audio driver, and in other embodiments, the first and second locations are adjacent each other. In some embodiments, at least a portion of the audio driver, flexible printed circuit, optical source and optical detector are encapsulated within a hydrophobic material.

An ear gel is attached to the body member, and includes opposite inner and outer surfaces. In some embodiments, the ear gel is configured to be removably secured to the body member. The ear gel is configured to be inserted within the ear canal of the subject and is transparent to one or more selected wavelengths of light or has one or more portions that are transparent to one or more selected wavelengths of light. A first light guide having opposite first and second ends is secured to the inner surface of the ear gel via the first end of the first light guide, and a second light guide having opposite first and second ends is secured to the inner surface of the ear gel via the first end of the second light guide. The second end of the first light guide is in optical communication with the optical source and the first light guide is configured to deliver light from the optical source into a first region of the ear canal. The second end of the second light guide is in optical communication with the optical detector and the second light guide is configured to collect light from a second region of the ear canal and deliver collected light to the optical detector.

According to other embodiments of the present invention, a sensor head assembly includes a body member sized to fit within an ear canal of a subject. The body member may have a generally cylindrical configuration, but other shapes may be utilized. The body member includes an inlet port and an exit port at respective opposite ends of the body member, and an internal acoustic passage extends between the inlet port and the exit port. In some embodiments, the acoustic passage extends along a longitudinal centerline of the body member. In other embodiments, at least a portion of the acoustic passage extends along a non-centerline portion of the body member. An audio driver is secured to the body member and is in acoustic communication with the acoustic passage of the body member. In some embodiments, the audio driver includes a sound outlet tube that is secured within the acoustic passage of the body member via the inlet port.

An optical source is secured to the audio driver, for example to the housing of the audio driver, at a first location and an optical detector is secured to the audio driver, for example to the housing of the audio driver, at a second location different from the first location. The optical source and optical detector are associated with a sensor, such as a PPG sensor, for monitoring/detecting physiological information from a person wearing the sensor head assembly. In some embodiments, the first and second locations are circumferentially spaced apart from each other relative to the audio driver, and in other embodiments, the first and second locations are adjacent each other. In some embodiments, at least a portion of the audio driver, optical source and optical detector are encapsulated within a hydrophobic material.

An ear gel is attached to the body member, and includes opposite inner and outer surfaces. In some embodiments, the ear gel is configured to be removably secured to the body member. The ear gel is configured to be inserted within the ear canal of the subject and is transparent to one or more selected wavelengths of light or has one or more portions that are transparent to one or more selected wavelengths of light. A first light guide having opposite first and second ends is secured to the inner surface of the ear gel via the first end of the first light guide, and a second light guide having opposite first and second ends is secured to the inner surface of the ear gel via the first end of the second light guide. The second end of the first light guide is in optical communication with the optical source and the first light guide is configured to deliver light from the optical source into a first region of the ear canal. The second end of the second light guide is in optical communication with the optical detector the second light guide is configured to collect light from a second region of the ear canal and deliver collected light to the optical detector.

In each of the above embodiments, the ear gel and the first and second light guides may comprise a flexible optically transmissive material having a durometer measurement of between Shore A10 and Shore A80. In each of the above embodiments, the ear gel may also include an opaque boundary configured to reduce optical cross-talk between the optical source and the optical detector.

According to other embodiments of the present invention, a hearing aid device includes a housing, a microphone disposed in the housing, and a sensor head assembly coupled to the housing. In some embodiments, the housing is adapted to be disposed behind an ear of the subject during operation of the hearing aid device. The sensor head assembly includes a body member sized to fit within an ear canal of a subject. The body member may have a generally cylindrical configuration, but other shapes may be utilized. The body member includes an inlet port and an exit port at respective opposite ends of the body member, and an internal acoustic passage extends between the inlet port and the exit port. In some embodiments, the acoustic passage extends along a longitudinal centerline of the body member. In other embodiments, at least a portion of the acoustic passage extends along a non-centerline portion of the body member.

An optical source is secured to the body member at a first location, and an optical detector is secured to the body member at a second location different from the first location. The optical source and optical detector are associated with a sensor, such as a PPG sensor, for monitoring/detecting physiological information from a person wearing the sensor head assembly. In some embodiments, the first and second locations are circumferentially spaced apart from each other relative to the body member, and in other embodiments, the first and second locations are adjacent each other. An audio driver is secured to the body member and is in acoustic communication with the acoustic passage of the body member. There is an electrical connection between the microphone and the audio driver when the sensor head assembly and housing are coupled together.

In some embodiments, the audio driver has a sound outlet tube, and the sound outlet tube is secured within the acoustic passage of the body member via the inlet port. In some embodiments, at least a portion of the audio driver, optical source and optical detector are encapsulated within a hydrophobic material.

An ear gel is attached to the body member, and includes opposite inner and outer surfaces. In some embodiments, the ear gel is configured to be removably secured to the body member. The ear gel is configured to be inserted within the ear canal of the subject and is transparent to one or more selected wavelengths of light or has one or more portions that are transparent to one or more selected wavelengths of light. A first light guide having opposite first and second ends is secured to the inner surface of the ear gel via the first end of the first light guide, and a second light guide having opposite first and second ends is secured to the inner surface of the ear gel via the first end of the second light guide. The second end of the first light guide is in optical communication with the optical source and the first light guide is configured to deliver light from the optical source into a first region of the ear canal. The second end of the second light guide is in optical communication with the optical detector the second light guide is configured to collect light from a second region of the ear canal and deliver collected light to the optical detector.

In some embodiments, the ear gel and the first and second light guides comprise a flexible optically transmissive material having a durometer measurement of between Shore A10 and Shore A80. In some embodiments, the ear gel includes an opaque boundary configured to reduce optical cross-talk between the optical source and the optical detector.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 illustrates a human ear with a sensor head assembly according to embodiments of the present invention inserted within the ear canal of the ear.

FIG. 2A is a cross-sectional view of the sensor head assembly of FIG. 1 within the ear canal.

FIG. 2B is an enlarged cross-sectional view of the sensor head assembly of FIG. 1 within the ear canal.

FIG. 3A is a front perspective view of the sensor head assembly of FIGS. 2A-2B with the internal components visible through the ear gel for clarity reasons.

FIG. 3B is a cross-sectional view of the sensor head assembly of FIG. 3A taken along lines 3B-3B.

FIG. 3C is a perspective view of the body member of the sensor head assembly of FIG. 3A illustrating the optical source and optical detector secured thereto at circumferentially spaced apart locations.

FIG. 4A is a front perspective view of a sensor head assembly according to other embodiments of the present invention and with the internal components visible through the ear gel for clarity reasons.

FIG. 4B is a cross-sectional view of the sensor head assembly of FIG. 4A taken along lines 4B-4B.

FIG. 5A is a front perspective view of a sensor head assembly according to other embodiments of the present invention and with the internal components visible through the ear gel for clarity reasons.

FIG. 5B is a cross-sectional view of the sensor head assembly of FIG. 5A taken along lines 5B-5B.

FIG. 5C is a front perspective view of a sensor head assembly according to other embodiments of the present invention and with the internal components visible through the ear gel for clarity reasons.

FIG. 5D is a cross-sectional view of the sensor head assembly of FIG. 5C taken along lines 5D-5D.

FIG. 6A is a front perspective view of a sensor head assembly according to other embodiments of the present invention and with the internal components visible through the ear gel for clarity reasons.

FIG. 6B is a cross-sectional view of the sensor head assembly of FIG. 6A taken along lines 6B-6B.

FIG. 7A is a front perspective view of a sensor head assembly according to other embodiments of the present invention and with the internal components visible through the ear gel for clarity reasons.

FIG. 7B is a cross-sectional view of the sensor head assembly of FIG. 7A taken along lines 7B-7B.

FIG. 8A illustrates a hearing aid device according to some embodiments of the present invention.

FIG. 8B illustrates the hearing aid device of FIG. 8A with the sensor head assembly inserted within an ear canal of a subject.

FIG. 9A is a perspective view of an ear gel having an opaque boundary or divider configured to reduce optical cross-talk between an optical source and an optical detector, according to some embodiments of the present invention.

FIG. 9B is a perspective view of an ear gel having an opaque boundary or divider configured to reduce optical cross-talk between an optical source and an optical detector, according to another embodiment of the present invention.

FIGS. 10A-10B illustrate the ear gel of FIGS. 9A-9B positioned within an auditory canal of a subject, respectively.

FIG. 11 illustrates a hearing aid device according to some embodiments of the present invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled”, or “secured” to another feature or element, it can be directly connected, attached, coupled, or secured to the other feature or element or intervening features or elements may be present. In contrast, when a feature or element is referred to as being “directly connected”, “directly attached”, “directly coupled”, or “directly secured” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

As used herein, the term “and/or” includes any and all combinations of one or more of the associated listed items and may be abbreviated as “/”.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features or elements, these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “about”, as used herein with respect to a value or number, means that the value or number can vary, for example, by as much as +/−20%.

The term “earbud”, as used herein, is intended to include any type of device or earpiece that may be attached to the ear (or ears) of a user and may have various configurations, without limitation.

The terms “optical source” and “optical emitter”, as used herein, are interchangeable.

The term “monitoring” refers to the act of measuring, quantifying, qualifying, estimating, sensing, calculating, interpolating, extrapolating, inferring, deducing, or any combination of these actions. More generally, “monitoring” refers to a way of getting information via one or more sensing elements. For example, “blood health monitoring” may include monitoring blood gas levels, blood hydration, and metabolite/electrolyte levels, etc.

The term “physiological” refers to matter or energy of or from the body of a creature (e.g., humans, animals, etc.). In embodiments of the present invention, the term “physiological” is intended to be used broadly, covering both physical and psychological matter and energy of or from the body of a creature. However, in some cases, the term “psychological” is called-out separately to emphasize aspects of physiology that are more closely tied to conscious or subconscious brain activity rather than the activity of other organs, tissues, or cells.

The term “body” refers to the body of a subject (human or animal) that may wear a device incorporating one or more optical sensor modules, according to embodiments of the present invention.

The term “coupling”, as used herein, refers to the interaction or communication between excitation light entering a region of a body and the region itself. For example, one form of optical coupling may be the interaction between excitation light generated from an optical sensor module and the blood vessels of the body of a user. In one embodiment, this interaction may involve excitation light entering the ear region and scattering from a blood vessel in the ear such that the intensity of scattered light is proportional to blood flow within the blood vessel.

The term “processor” is used broadly to refer to a signal processor or computing system or processing or computing method which may be localized or distributed. For example, a localized signal processor may comprise one or more signal processors or processing methods localized to a general location, such as to an earbud. Examples of a distributed processor include “the cloud”, the internet, a remote database, a remote processor computer, a plurality of remote processors or computers in communication with each other, or the like, or processing methods distributed amongst one or more of these elements. The key difference is that a distributed processor may include delocalized elements, whereas a localized processor may work independently of a distributed processing system. As a specific example, microprocessors, microcontrollers, ASICs (application specific integrated circuits), analog processing circuitry, or digital signal processors are a few non-limiting examples of physical signal processors that may be found in wearable devices.

The term “remote” does not necessarily mean that a remote device is a wireless device or that it is a long distance away from a device in communication therewith. Rather, the term “remote” is intended to reference a device or system that is distinct from another device or system or that is not substantially reliant on another device or system for core functionality. For example, a computer wired to a wearable device may be considered a remote device, as the two devices are distinct and/or not substantially reliant on each other for core functionality. However, any wireless device (such as a portable device, for example) or system (such as a remote database for example) is considered remote to any other wireless device or system.

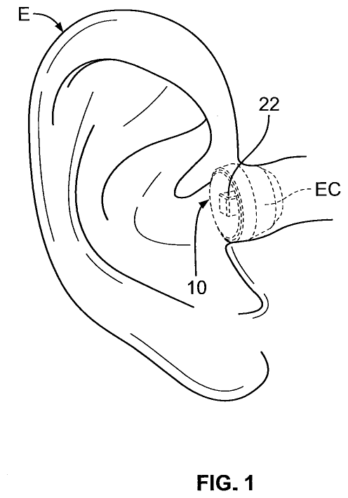

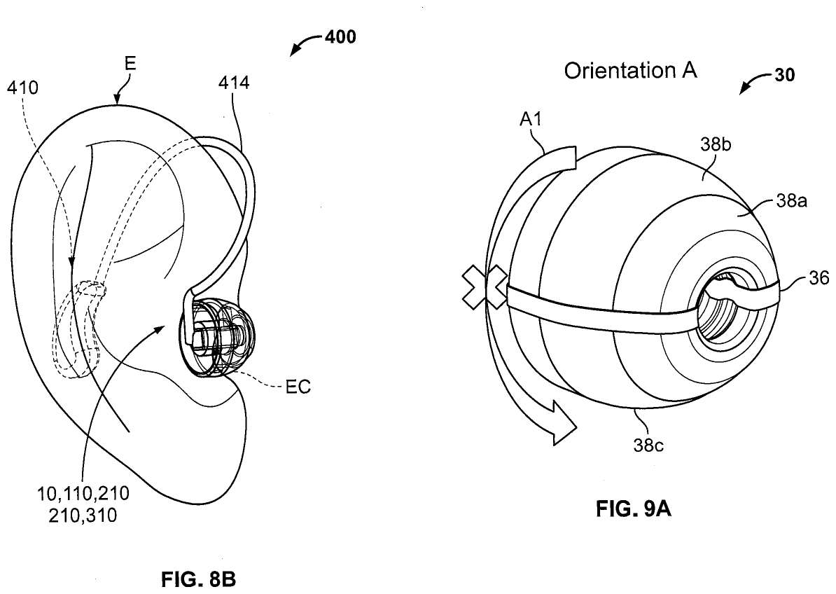

Referring now to the drawings, embodiments of the present invention will be illustrated. FIG. 1 illustrates a human ear E with a sensor head assembly 10 comprising an audio driver 22, according to embodiments of the present invention, inserted within the ear canal EC of the ear E. The sensor head assembly may be part of a hearing aid device (400, FIGS. 8A-8B) and configured to be coupled to a housing (410, FIGS. 8A-8B) containing a power supply. The housing 410 may also include various additional electronic components including, but not limited to, a signal processor, a wireless module for communicating with a remote device, a microphone, a speaker, an environmental sensor, a memory storage device, etc. Moreover, a battery (such as a lithium polymer battery or other portable battery) or other power source (such as an energy harvesting source) may be mounted within the housing 410. The power source may be charged via a charge port, such as a USB charge port, for example. When the sensor head assembly 10 and housing 410 are coupled together, an electrical connection is established between the power source and the audio driver 22 of the sensor head assembly 10.

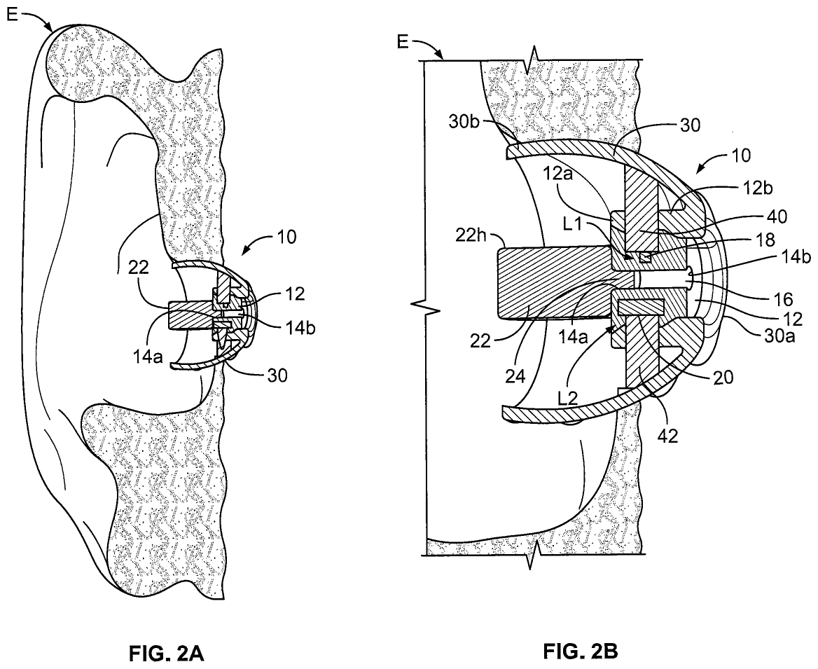

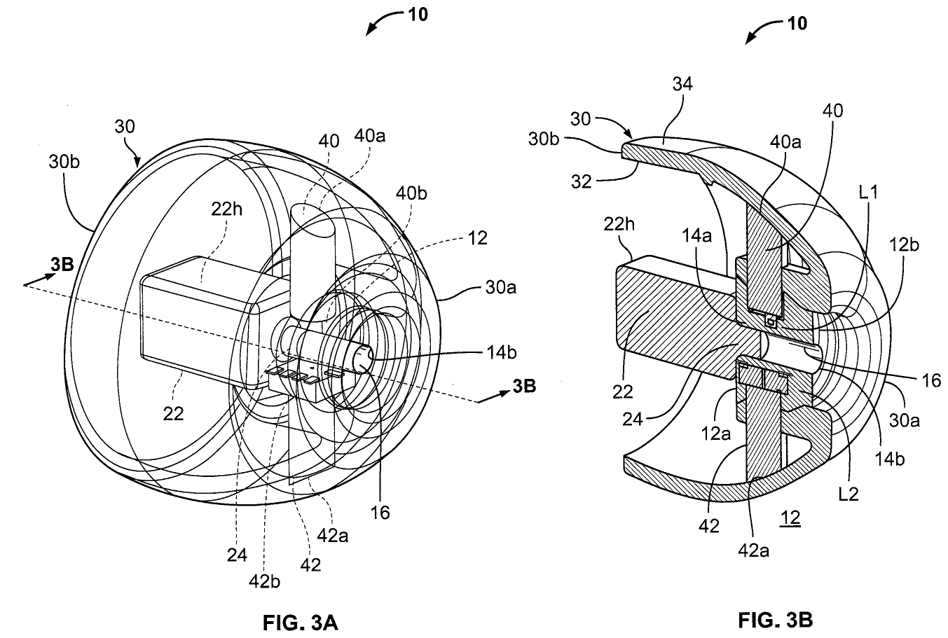

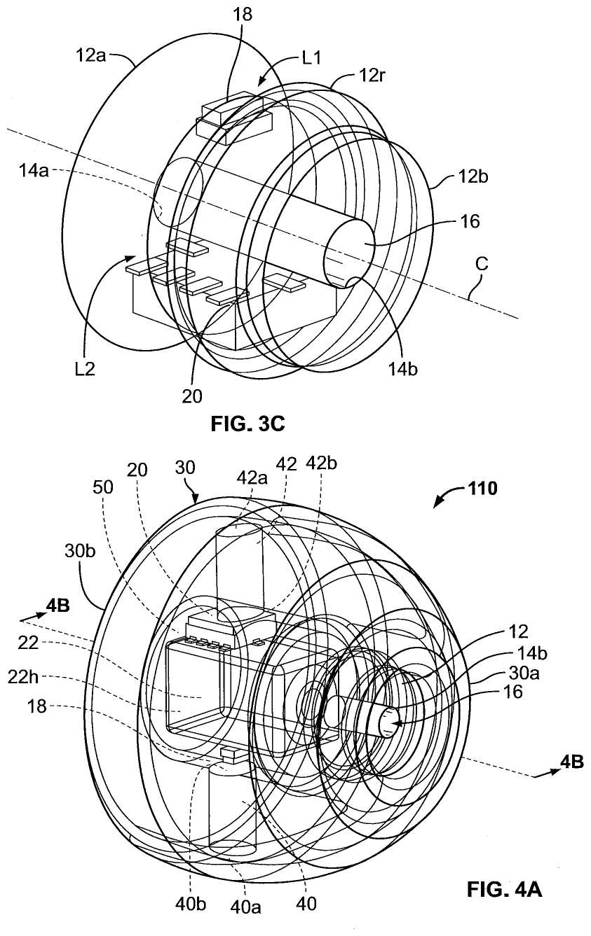

Referring to FIGS. 2A-2B and 3A-3C, a sensor head assembly 10, according to a first embodiment of the present invention, is illustrated. The illustrated sensor head assembly 10 includes a body member 12 sized to fit within an ear canal of a subject. The body member 12 includes an inlet port 14a and an exit port 14b at respective opposite ends 12a, 12b of the body member 12. The illustrated body member 12 has a generally cylindrical configuration, although other shapes may be utilized. A key benefit of a cylindrical configuration is that it may be more conforming to the shape of a person's ear canal and thus more comfortable to wear long-term. However, a rectangular configuration or other configuration may be used.

An internal acoustic passage 16 extends between the inlet port 14a and the exit port 14b that is configured to direct sound from the audio driver 22 to the ear of a wearer of a device incorporating the sensor head assembly 10. In the illustrated embodiment, the acoustic passage 16 extends along a longitudinal centerline C (FIG. 3C) of the body member 12. In other embodiments, at least a portion of the acoustic passage may extend along a non-centerline portion of the body member 12.

In some embodiments the body member 12 is a plastic member made from polycarbonate or a reinforced plastic such as a glass filled polyarylamide (e.g., IXEF® brand polyarylamide available from Solvay Group of Belgium). However, the body member 12 may be formed from various other materials. For example, the body member 12 may be formed from a metal or metallic material. The body member 12 may be opaque which serves to reduce optical cross-talk between an optical source 18 and an optical detector 20, particularly when the optical source 18 and an optical detector 20 are circumferentially spaced apart from each other on the body member 12, as described below.

An optical source 18 is secured to or integrated within the body member 12 at a first location L1, and an optical detector 20 is secured to or integrated within the body member 12 at a second location L2 different from the first location L1. The optical source 18 and optical detector 20 are associated with a sensor, such as a PPG sensor, for monitoring/detecting physiological information from a person wearing the sensor head assembly 10. In the illustrated embodiment of FIGS. 2A-2B and 3A-3C, the first and second locations L1, L2 of the optical source 18 and the optical detector 20 are circumferentially spaced apart from each other on the body member 12. Specifically, the first and second locations L1, L2 are diametrically opposite each other (i.e., 180° apart) in the illustrated embodiment. However, the optical source 18 and optical detector 20 may be circumferentially spaced apart from each other by various amounts (e.g., between 0° and 180° apart). For example, the first and second locations L1, L2 may be 90° apart, 60° apart, 45° apart, 30° apart, etc. In some embodiments, the first and second locations L1, L2 may be adjacent each other on the body member 12 (i.e., 0° apart). In other words, the optical source 18 and the optical detector 20 may be positioned adjacent each other at a location on the body member 12.

Electrical connections are provided within the body member 12 to create a functional circuit to power the optical source 18 and the optical detector 20. Such electrical connections/circuitry could include sheet metal geometry that is molded within the polymeric material of the body member 12, a race track to accept a flexible printed circuit, a secondary operation such as electro or electroless plating to deposit conductive metal, discrete wires, and the like, any of which are attached to the optical source 18 and the optical detector 20 to complete the electrical circuit.

In some embodiments, at least a portion of the audio driver 22, optical source 18 and optical detector 20 are encapsulated within a hydrophobic encapsulant material H.

The optical source 18 may be one or more light-emitting diodes (LED), laser diodes (LD), compact incandescent bulbs, micro-plasma emitters, IR blackbody sources, organic LEDs, or the like. The optical detector 20 may be one or more photodiodes, photodetectors, phototransistors, thyristors, solid state devices, optical chipsets, or the like.

An audio driver 22 is secured to the body member 12 and is in acoustic communication with the acoustic passage 16 of the body member 12. The illustrated audio driver 22 has a housing 22h with a generally rectangular configuration and a sound outlet tube 24 extending outward from an end portion of the housing 22h. However, audio drivers that may be utilized in accordance with embodiments of the present invention may have various shapes and configurations and are not limited to the illustrated shape/configuration.

The sound outlet tube 24 is inserted within the acoustic passage 16 of the body member 12 via the inlet port 14a. The audio driver 22 may be secured to the body member 12 via a snug fit of the sound outlet tube 24 within the acoustic passage 16. In other embodiments, a snap fit configuration may be utilized. Non-limiting exemplary audio drivers that may be utilized with embodiments of the present invention are available from Knowles acoustic Components, Itasca Ill.

An ear gel 30 is attached to the body member 12, and includes opposite inner and outer surfaces 32, 34, and opposite first and second end portions 30a, 30b. The ear gel 30 is configured to be inserted within the ear canal EC of a subject and is transparent to one or more selected wavelengths of light or has one or more portions that are transparent to one or more selected wavelengths; alternately, the ear gel 30 comprises at least one window (e.g., an opening or an optically transparent region) for the optical emitter or optical detector to be exposed to the ear canal of the subject. The ear gel 30 is formed of a soft, conformable material, such as silicone, and may have a substantially uniform wall thickness between the inner surface 32 and the outer surface 34. However, variable wall thicknesses may be utilized in some embodiments. One benefit of variable wall thicknesses may be for the purpose of controlling the light-guiding properties of the ear gel 30. For example, by thickening the ear gel 30 in some regions and thinning the ear gel 30 in other regions, the desired light-guiding may be better confined to either the thicker or thinner regions, depending upon the desired wavelength or polarization of light that may be emitted the optical emitter 18 or detected by the optical detector 20.

Material for the ear gel 30 is not limited to silicone; various other soft, thermoplastic elastomers that are at least partially light-transparent, may be used, also. Although a hard ear gel 30 may be used within the invention, a non-compliant ear gel may generate a pain response in the person wearing the device.

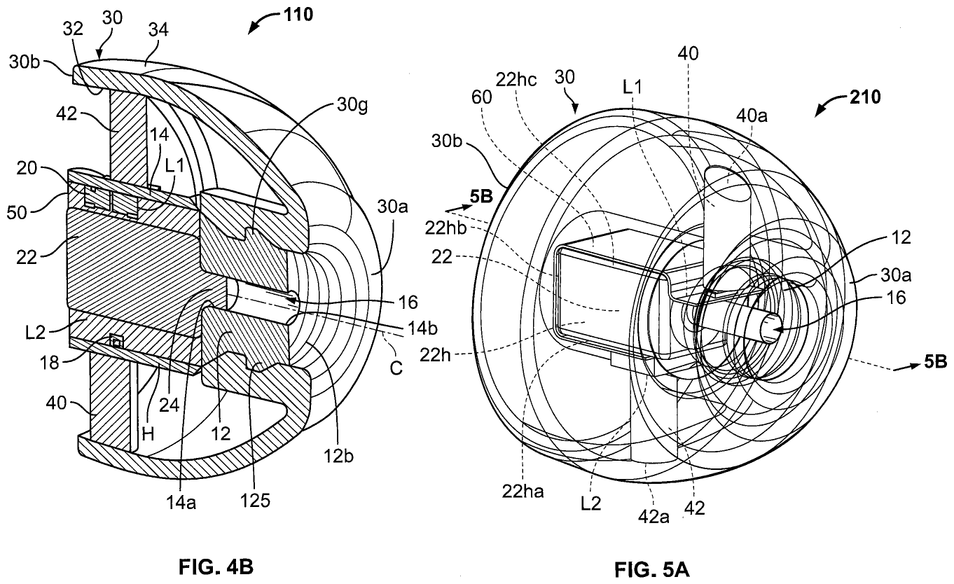

In some embodiments, the ear gel 30 is designed to be replaceable and can be removably secured to the body member 12 such that a user can select and use an ear gel 30 with a shape and/or size that best fits the ear of the user. For example, as illustrated in FIG. 4B, the ear gel 30 has a geometry and configuration that interlocks with a corresponding geometry and configuration of the body member 12. In the illustrated embodiment of in FIG. 4B, the body member 12 includes an annular ridge 12r that is configured to be inserted within an annular receiving groove 30g of the ear gel 30. The annular ridge 12r of the body member 12 and the annular receiving groove 30g of the ear gel 30 can have various shapes and configurations and are not limited to the illustrated configuration. The interlocking geometry of the ear gel 30 and body member 12 serve to constrain the ear gel 30 to the body member 12, yet allow the ear gel 30 to be removable.

A first light guide 40 having opposite first and second ends 40a, 40b is secured to or integrally formed with the inner surface 32 of the ear gel 30 via the first end 40a, and a second light guide 42 having opposite first and second ends 42a, 42b is secured to or integrally formed with the inner surface 32 of the ear gel 30 via the first end 42a. In some embodiments, the light guides 40, 42 are molded with the ear gel 30 such that the ear gel 30 and the light guides 40, 42 are an integral unit. The first and second light guides 40, 42 may be formed of a soft, conformable material, such as silicone, and may be configured to be malleable with the ear gel 30 when the sensor head assembly 10 is secured within the ear canal of a user. In other words, the light guides 40, 42 may be conformable with the ear gel 30 as the ear gel 30 is pressed into the ear canal of an ear. Material for the light guides 40, 42 is not limited to silicone; various other soft, thermoplastic elastomers that are light-transparent, may be used, also.

It should also be noted that in some embodiments, the light guides 40, 42 may be rigid. Additionally, in some embodiments the light guides 40, 42 may extend beyond the ear gel 30 and not conform to the shape of the ear gel 30. In such case, the distal ends 40a, 42a of the light guides 40, 42 should preferably have a diameter greater than about 3mm such that they do not cause discomfort to someone wearing the sensor head assembly 10.

The second end 40b of the first light guide 40 is positioned so as to be in optical communication with the optical source 18 such that the first light guide 40 can deliver light from the optical source into a first region of the ear canal through a portion of the ear gel 30. The second end 42b of the second light guide is positioned so as to be in optical communication with the optical detector such that the second light guide 42 can collect light from a second region of the ear canal through a portion of the ear gel 30 and deliver collected light to the optical detector 20. In some embodiments, the ear gel 30 and body member 12 may be configured such that, when the ear gel 30 is secured to the body member 12, the second (or distal) ends 40b, 42b of the light guides 40, 42 align to form a butt joint with respective emitting and detecting surfaces of the optical source 18 and the optical detector 20.

In some embodiments of the present invention, the ear gel 30 and the first and second light guides 40, 42 are formed from a flexible optically transmissive material having a durometer measurement of between Shore A10 and Shore A80. In some embodiments, the ear gel 30 may include an opaque boundary configured to reduce optical cross-talk between the optical source 18 and the optical detector 20. In some embodiments of the present invention, all of the ear gel 30 and light guides 40, 42 can be manufactured via a single-shot mold using silicone (or other material) having the same durometer. Alternatively, the ear gel 30 and light guides 40, 42 can have different durometer values, as a result of using different manufacturing methods and/or materials.

In some embodiments of the present invention, one or both of the light guides 40, 42 may have a generally cylindrical configuration. In other embodiments, one or both of the light guides 40, 42 may have a generally non-cylindrical configuration, e.g., rectangular, triangular, oval, etc.

In some embodiments, an optical filter may be integrated within one or more of the light guides 40, 42. For example, a light guide 40, 42 may comprise a material having an optically filtering dye or a material which inherently filters one or more wavelengths of light. As one example, either or both of the light guides 40, 42 may comprise, wholly or partially, a dye therewithin. As one specific example, a dye, such as an infrared dye designed to block visible wavelengths but pass IR wavelengths may be utilized. For example, a polycarbonate or acrylic light guide, dyed with Filtron® absorptive dye E800 (Gentex Corporation, Carbondale, Pa.), would facilitate both light-guiding and IR-pass filtering functionality. Alternatively, another example of such an integrated physical optical filter comprises absorptive dyes available from Sabic (Riyadh, Saudi Arabia) dispersed in polycarbonate and/or acrylic to create an edge or long-pass optical filter. At least one of the light guides 40, 42 may be partially or wholly comprised of such a material, thereby facilitating the combinational purpose of light guiding and optical filtering. A few additional non-limiting examples of an inherently filtering material includes sapphire, which absorbs some infrared (IR) wavelengths, and glass, which absorbs some ultraviolet (UV) wavelengths. However, various types of filtering material may be utilized, without limitation.

In some embodiments, a physical optical filter can be disposed within the ear gel 30 at or near the location where the first ends 40a, 42a of the light guides 40, 42 are attached to the inner surface 32 of the ear gel 30.

In some embodiments, an optical filter may be integrated with the optical source 18 and/or the optical detector 20. For example, a bandpass filter, such as an interference filter or the like, may be disposed on the top of the optical source 18 and/or optical detector 20. Alternatively (or additionally), an optical filter effect may be integrated within the semiconductor material comprising the optical source 18 and/or optical detector 20, such as by selective ion implantation of certain regions within silicon or by band-gap engineering within compound semiconductors, such as the AlInGaAs or AlInGaN system of semiconductor engineering.

In some embodiments, one or both of the light guides 40, 42 may be surrounded or partially surrounded by a cladding/barrier material that is configured to at least partially block light from an external source from entering the light guides 40, 42 at select locations along the light guides and/or at least partially confine light within the light guides 40, 42 and to support light-guiding along the longitudinal axis of the light guides 40, 42. The cladding/barrier material may be a light blocking material and/or a light reflective material and/or a material that has a higher optical scattering coefficient than the light guiding material of the light guides 40, 42. For example, the cladding material may be a dark (e.g., black, etc.) or silver (or other reflective color) coating, a material with refractive index that is smaller than that of the core light guide material. In some embodiments, the cladding material may be the supporting material itself (such as silicone or whatever the ear gel 30 is formed from).

In some embodiments, the cladding material may be a reflective material between the light guides 40, 42. In some cases the reflective material may surround the outer surface of the light guides 40, 42 so that only the ends 40a, 40b, 42a, 42b of the light guides 40, 42 are exposed to the light pathway. The reflective material may be mylar, metallic material, a roughened texture, or the like.

In some embodiments, the light guides 40, 42 may comprise a roughened surface along the periphery of the light guides 40, 42 configured to scatter light within the light guide 40, 42. In some embodiments, the light guides 40, 42 may comprise a roughened surface along the distal ends 40a, 42a and/or proximal ends 40b, 42b configured to diffuse light.

In some embodiments of the present invention, the light-guiding material of one or more of the light guides 40, 42 may comprise polarizing material. Exemplary polarizing material that can be used in accordance with embodiments of the present invention is available from American Polarizers, Inc., Reading, Pennsylvania, as well as Edmund Optics, Barrington, N.J. A key benefit of a cross-polarizing implementation, where the optical emitter polarizer is configured to be orthogonally polarized with respect to the optical detector polarizer, may be that unwanted specular reflection is attenuated such that the light beam collected by the optical detector comprises a higher percentage of photons that have passed through a blood flow region of the body.

Improved optical coupling may be achieved by the ear gel 30 because of the soft, conformable material of the ear gel 30 that increases in surface area when acted upon by the surrounding anatomy of the ear canal. For example, when the outer surface 34 of the ear gel 30 rests upon a planar surface, point contact exists. However, when the ear gel 30 is acted upon by the surrounding geometry of the ear (i.e., non-planar surface), the ear gel 30 deforms along with the ends 40a, 42a of the light guides 40, 42 and point contact may adapt to mimic the area of the surrounding ear anatomy. Point contact, thus, increases to area contact. However, the dimensions of the flexible light guides 40, 42 attached to the ear gel 30 themselves are not greatly affected by motion and hence do not generate a significant amount of motion artifacts during physical activity of the user wearing a device incorporating the sensor head assembly 10.

The ends 40a, 42a of the light guides 40, 42 (as well as the light guides 40, 42 themselves) are preferably soft and compliant such that a deformation (i.e., change of shape as a result of being inserted in an ear) of the ear gel 30 at the vicinity of the light guides 40, 42, causes the light guides 40, 42 to also deform, but without contributing to motion artifacts within light passing through the light guides 40, 42. The soft, compliant nature of the light guides 40, 42 may improve optical coupling between the light guides 40, 42 and the ear of a user because of the reduced susceptibility to motion. For example, a portion of each light guide 40, 42 within the ear gel 30 changes shape (i.e., deforms) with the ear gel 30 so as not to become optically decoupled from the ear. The increased optical coupling may increase the ratio of desired modulated signal (the blood flow signal) from the total optical signal (i.e., provide a higher signal-to-noise ratio). It may also serve to further reduce movement of the sensor head assembly 10 as the additional surface area increases friction.

Referring to FIGS. 4A-4B, 5A-5B, 5C-5D, 6A-6B and 7A-7B, sensor head assemblies 110, 210, 210′, 310 according to additional embodiments of the present invention, are illustrated. For example, referring to FIGS. 4A-4B, a sensor head assembly 110 according to a second embodiment of the present invention includes a body member 12 sized to fit within an ear canal of a subject. The body member 12 includes an inlet port 14a and an exit port 14 at respective opposite ends 12a, 12b of the body member 12. The illustrated body member 12 has a generally cylindrical configuration, although other shapes may be utilized. An internal acoustic passage 16 extends between the inlet port 14a and the exit port 14b that is configured to direct sound from the audio driver 22 to the ear of a wearer of a device incorporating the sensor head assembly 10. In the illustrated embodiment, the acoustic passage 16 extends along a longitudinal centerline C of the body member 12. In other embodiments, at least a portion of the acoustic passage may extend along a non-centerline portion of the body member 12.

An audio driver 22 as described above is secured to the body member 12 and is in acoustic communication with the acoustic passage 16 of the body member 12. The illustrated audio driver 22 has a housing 22h with a generally rectangular configuration and a sound outlet tube 24 extending outward from an end portion of the housing 22h. However, audio drivers that may be utilized in accordance with embodiments of the present invention may have various shapes and configurations and are not limited to the illustrated shape/configuration. The sound outlet tube 24 is inserted within the acoustic passage 16 of the body member 16 via the inlet port 14a. The audio driver 22 may be secured to the body member via a snug fit of the sound outlet tube 24 within the acoustic passage 16. In other embodiments, a snap fit configuration may be utilized.

An annular member 50 surrounds at least a portion of the audio driver 22. An optical source 18 is secured to or integrated within the annular member 50 at a first location L1, and an optical detector 20 is secured to or integrated within the annular member 50 at a second location L2 different from the first location. The optical source 18 and optical detector 20 are associated with a sensor, such as a PPG sensor, for monitoring/detecting physiological information from a person wearing the sensor head assembly 10. In the illustrated embodiment of FIGS. 4A-4B, the first and second locations L1, L2 of the optical source 18 and the optical detector 20 are circumferentially spaced apart from each other on the annular member 50. Specifically, the first and second locations L1, L2 are diametrically opposite each other (i.e., 180° apart) in the illustrated embodiment. However, the optical source 18 and optical detector 20 may be circumferentially spaced apart from each other by various amounts (e.g., between 0° and 180° apart). For example, the first and second locations L1, L2 may be 90° apart, 60° apart, 45° apart, 30° apart, etc. In some embodiments, the first and second locations L1, L2 may be adjacent each other on the annular member 50 (i.e., 0° apart). In other words, the optical source 18 and the optical detector 20 may be positioned adjacent each other at a location on the annular member 50.

An ear gel 30 as described above is attached to the body member 12, and includes opposite inner and outer surfaces 32, 34, and opposite first and second end portions 30a, 30b. The ear gel 30 is configured to be inserted within the ear canal EC of a subject and is transparent to one or more selected wavelengths of light or has one or more portions that are transparent to one or more selected wavelengths. First and second light guides 40, 42, as described above, are attached to the inner surface 32 of the ear gel 30. Specifically, a first light guide 40 having opposite first and second ends 40a, 40b is secured to or integrally formed with the inner surface 32 of the ear gel 30 via the first end 40a, and a second light guide 42 having opposite first and second ends 42a, 42b is secured to or integrally formed with the inner surface 32 of the ear gel 30 via the first end 42a. The second end 40b of the first light guide 40 is positioned so as to be in optical communication with the optical source 18 such that the first light guide 40 can deliver light from the optical source into a first region of the ear canal through a portion of the ear gel 30. The second end 42b of the second light guide is positioned so as to be in optical communication with the optical detector such that the second light guide can collect light from a second region of the ear canal through a portion of the ear gel 30 and deliver collected light to the optical detector 20. In some embodiments, the ear gel 30 and body member 12 may be configured such that, when the ear gel 30 is secured to the body member 12, the second (or distal) ends 40b, 42b of the light guides 40, 42 align to form a butt joint with respective emitting and detecting surfaces of the optical source 18 and the optical detector 20.

In the illustrated embodiment, the audio driver 22, annular member 50, optical source 18 and optical detector 20 are encapsulated within a hydrophobic encapsulant material H.

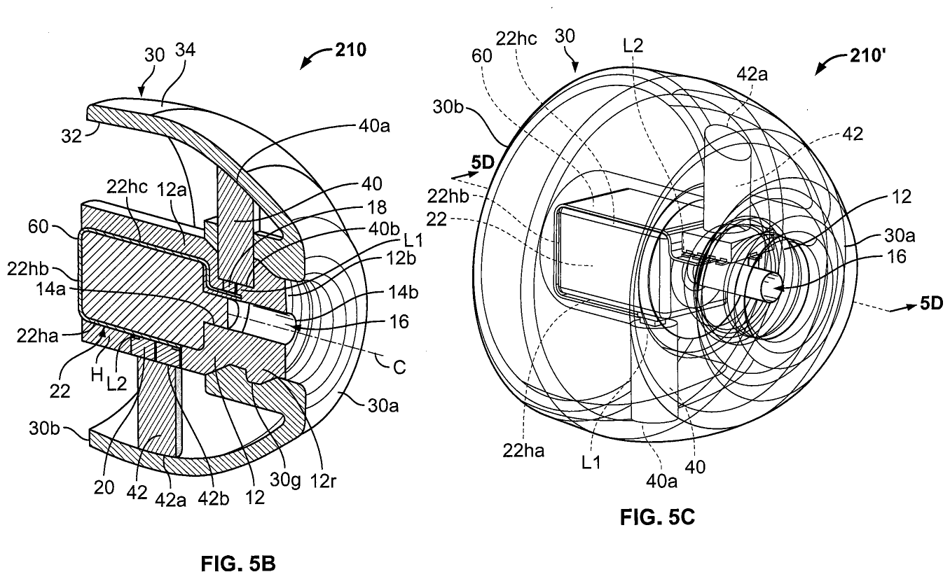

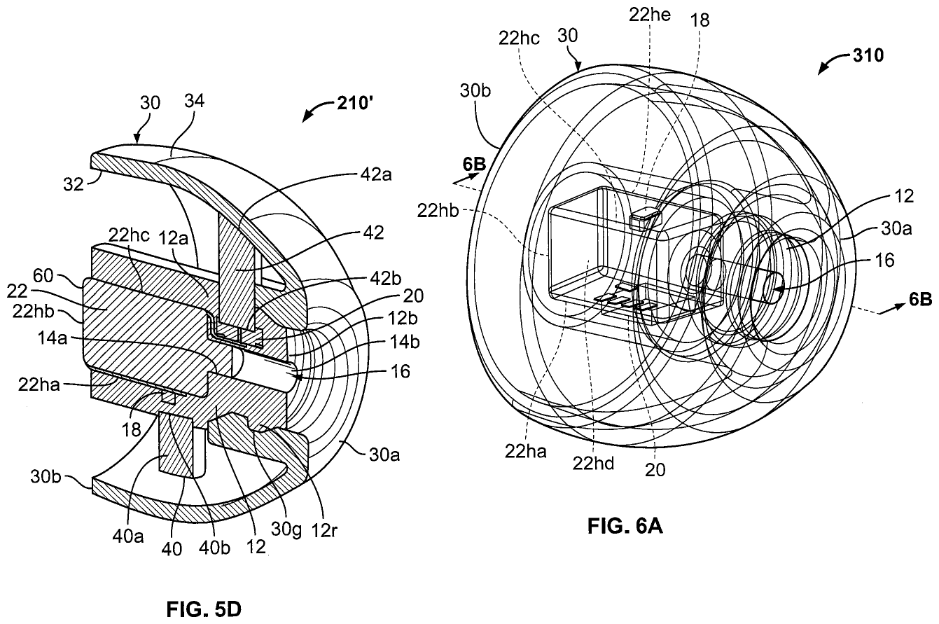

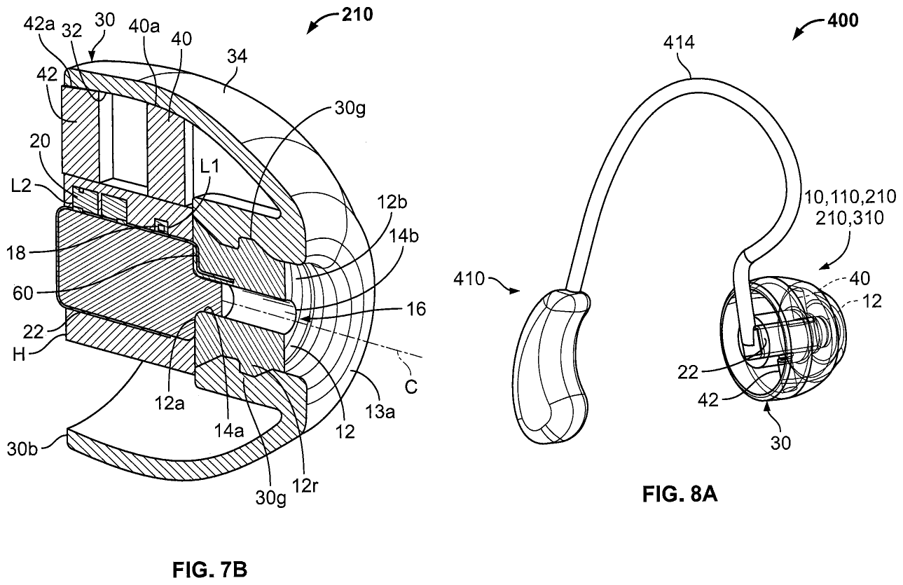

Referring to FIGS. 5A-5B and 7A-7B, a sensor head assembly 210 according to a third embodiment of the present invention includes a body member 12 sized to fit within an ear canal of a subject. The body member 12 includes an inlet port 14a and an exit port 14 at respective opposite ends 12a, 12b of the body member 12. The illustrated body member 12 has a generally cylindrical configuration, although other shapes may be utilized. An internal acoustic passage 16 extends between the inlet port 14a and the exit port 14b that is configured to direct sound from the audio driver 22 to the ear of a wearer of a device incorporating the sensor head assembly 10. In the illustrated embodiment, the acoustic passage 16 extends along a longitudinal centerline C of the body member 12. In other embodiments, at least a portion of the acoustic passage may extend along a non-centerline portion of the body member 12.

An audio driver 22 as described above is secured to the body member 12 and is in acoustic communication with the acoustic passage 16 of the body member 12. The illustrated audio driver 22 has a housing 22h with a generally rectangular configuration and a sound outlet tube 24 extending outward from an end portion of the housing 22h. However, audio drivers that may be utilized in accordance with embodiments of the present invention may have various shapes and configurations and are not limited to the illustrated shape/configuration. The sound outlet tube 24 is inserted within the acoustic passage 16 of the body member 16 via the inlet port 14a. The audio driver 22 may be secured to the body member via a snug fit of the sound outlet tube 24 within the acoustic passage 16. In other embodiments, a snap fit configuration may be utilized.

A flexible printed circuit 60 is secured to the audio driver 22. In the illustrated embodiment the flexible printed circuit 60 extends around an outer surface of the housing 22h of the audio driver. More specifically, the flexible printed circuit 60 is attached to first, second and third surfaces 22ha, 22hb, 22hc of the audio driver housing 22h. An optical source 18 is secured to the flexible printed circuit 60 at a first location L1, and an optical detector 20 is secured to the flexible printed circuit 60 at a second location L2 different from the first location L1 on the flexible printed circuit 60. The optical source 18 and optical detector 20 are associated with a sensor, such as a PPG sensor, for monitoring/detecting physiological information from a person wearing the sensor head assembly 10. In the illustrated embodiment of FIGS. 5A-5B, the first and second locations L1, L2 of the optical source 18 and the optical detector 20 are on opposite sides of the audio driver housing 22h. However, in other embodiments as illustrated in FIGS. 7A-7B, the optical source 18 and optical detector 20 may be positioned adjacent each other on the flexible printed circuit 60 (i.e., the first and second locations L1, L2 are adjacent each other on the same side of the audio driver housing 22h). Although a rigid circuit board may be used within embodiments of the present invention, a flexible printed circuit board may enable a smaller overall earpiece (i.e., sensor head assembly), which is particularly important for a hearing aid device.

In the illustrated embodiment, the flexible printed circuit 60, audio driver 22, optical source 18 and optical detector 20 are encapsulated within a hydrophobic encapsulant material H.

An ear gel 30 as described above is attached to the body member 12, and includes opposite inner and outer surfaces 32, 34, and opposite first and second end portions 30a, 30b. The ear gel 30 is configured to be inserted within the ear canal EC of a subject and is transparent to one or more selected wavelengths of light or has one or more portions that are transparent to one or more selected wavelengths. First and second light guides 40, 42, as described above, are attached to the inner surface 32 of the ear gel 30. Specifically, a first light guide 40 having opposite first and second ends 40a, 40b is secured to or integrally formed with the inner surface 32 of the ear gel 30 via the first end 40a, and a second light guide 42 having opposite first and second ends 42a, 42b is secured to or integrally formed with the inner surface 32 of the ear gel 30 via the first end 42a. The second end 40b of the first light guide 40 is positioned so as to be in optical communication with the optical source 18 such that the first light guide 40 can deliver light from the optical source into a first region of the ear canal through a portion of the ear gel 30. The second end 42b of the second light guide is positioned so as to be in optical communication with the optical detector such that the second light guide can collect light from a second region of the ear canal through a portion of the ear gel 30 and deliver collected light to the optical detector 20. In some embodiments, the ear gel 30 and body member 12 may be configured such that, when the ear gel 30 is secured to the body member 12, the second (or distal) ends 40b, 42b of the light guides 40, 42 align to form a butt joint with respective emitting and detecting surfaces of the optical source 18 and the optical detector 20.

Referring to FIGS. 5C-5D, a sensor head assembly 210′ according to a fourth embodiment of the present invention is illustrated. The illustrated embodiment of FIGS. 5C-5D is essentially identical to the embodiment of FIGS. 5A-5B except that the locations of the optical source 18 and the optical detector 20 are reversed. In FIGS. 5A-5B, the optical source 18 and first light guide 40 are positioned closer to the first end 30a of the ear gel 30 than the optical detector 20 and the second light guide 42. In FIGS. 5C-5D, the optical detector 20 and second light guide 42 are positioned closer to the first end 30a of the ear gel 30 than the optical source 18 and the first light guide 40.

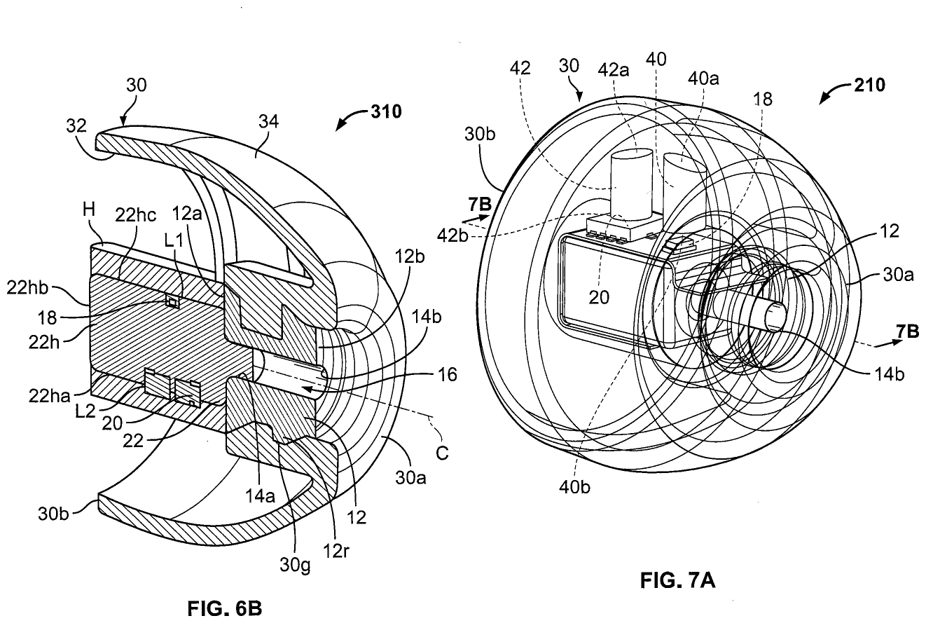

Referring to FIGS. 6A-6B, a sensor head assembly 310 according to a fifth embodiment of the present invention includes a body member 12 sized to fit within an ear canal of a subject. The body member 12 includes an inlet port 14a and an exit port 14 at respective opposite ends 12a, 12b of the body member 12. The illustrated body member 12 has a generally cylindrical configuration, although other shapes may be utilized. An internal acoustic passage 16 extends between the inlet port 14a and the exit port 14b that is configured to direct sound from the audio driver 22 to the ear of a wearer of a device incorporating the sensor head assembly 10. In the illustrated embodiment, the acoustic passage 16 extends along a longitudinal centerline C of the body member 12. In other embodiments, at least a portion of the acoustic passage may extend along a non-centerline portion of the body member 12.

An audio driver 22 as described above is secured to the body member 12 and is in acoustic communication with the acoustic passage 16 of the body member 12. The illustrated audio driver 22 has a housing 22h with a generally rectangular configuration and a sound outlet tube 24 extending outward from an end portion of the housing 22h. However, audio drivers that may be utilized in accordance with embodiments of the present invention may have various shapes and configurations and are not limited to the illustrated shape/configuration. The sound outlet tube 24 is inserted within the acoustic passage 16 of the body member 16 via the inlet port 14a. The audio driver 22 may be secured to the body member via a snug fit of the sound outlet tube 24 within the acoustic passage 16. In other embodiments, a snap fit configuration may be utilized.

An optical source 18 is secured to or integrated within the housing 22h of the audio driver 22 at a first location L1, and an optical detector 20 is secured to or integrated within the housing 22h of the audio driver 22 at a second location L2 different from the first location. The optical source 18 and optical detector 20 are associated with a sensor, such as a PPG sensor, for monitoring/detecting physiological information from a person wearing the sensor head assembly 10. In the illustrated embodiment of FIGS. 6A-6B, the first and second locations L1, L2 of the optical source 18 and the optical detector 20 are on opposite sides of the audio driver housing 22h. However, in other embodiments, the optical source 18 and optical detector 20 may be positioned adjacent each other on the same side of the audio driver housing 22h. Moreover, in other embodiments the optical source 18 and optical detector 20 may be positioned on either of the other sides 22hd, 22he of the audio driver housing 22h (i.e., adjacent each other on a side 22hd or 22he, or positioned opposite each other on respective sides 22hd, 22he).

An ear gel 30 as described above is attached to the body member 12, and includes opposite inner and outer surfaces 32, 34, and opposite first and second end portions 30a, 30b. The ear gel 30 is configured to be inserted within the ear canal EC of a subject and is transparent to one or more selected wavelengths of light or has one or more portions that are transparent to one or more selected wavelengths. First and second light guides (not illustrated) can be attached to the inner surface 32 of the ear gel 30 so as to be in optical communication with the optical source 18 and optical detector 20, as described above.

Referring to FIGS. 8A-8B, a hearing aid device 400 includes a housing 410 and a sensor head assembly 10, 110, 210, 210′, 310 as described above coupled to the housing 410. The housing 410 is electrically connected to the sensor head assembly 10, 110, 210, 210′, 310 via cable 414. The cable 414 may be electrical wiring surrounded by a protective sheath, which may help prevent the electrical wiring from coming in unwanted contact with the body, moisture, or the like. In some embodiments, the housing 410 is adapted to be disposed behind an ear E of a user during operation of the hearing aid device, as illustrated in FIG. 8B.

In some embodiments, the housing 410 may comprise at least one biometric sensor, such as a PPG sensor, ECG sensor, inertial sensor, auscultatory sensor, or the like. In some embodiments, the housing 410 may comprise at least one environmental sensor, such as an ambient light sensor, humidity sensor, temperature sensor, or the like.

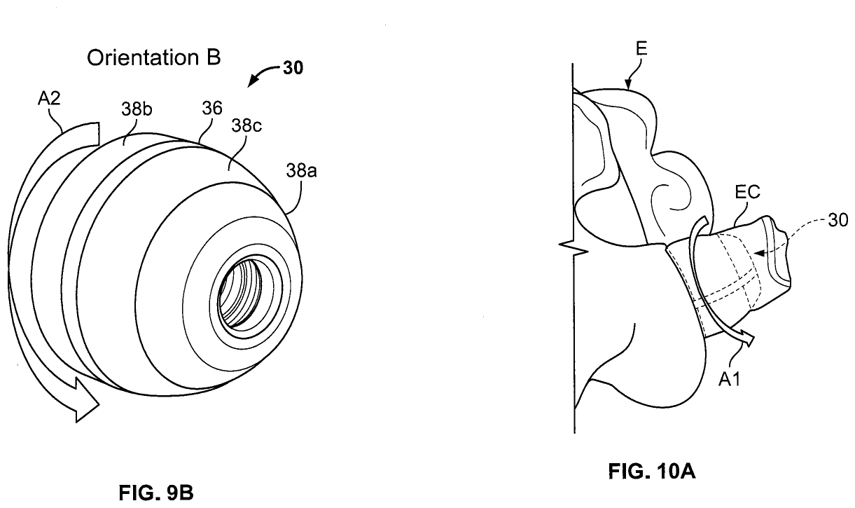

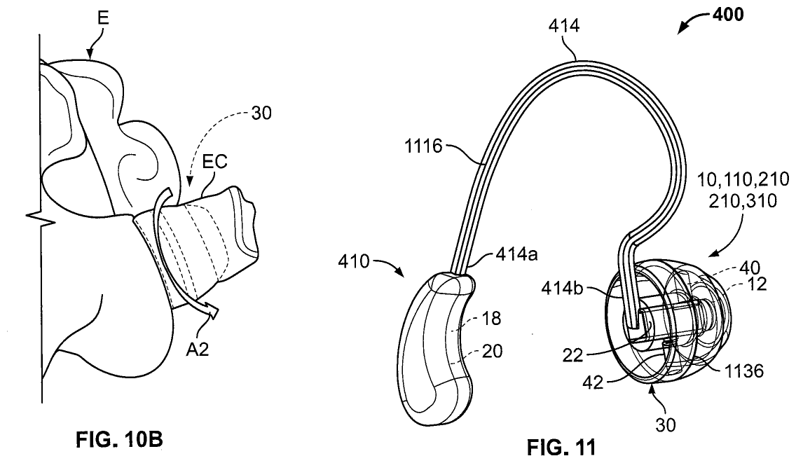

Referring to FIGS. 9A-9B and 10A-10B, ear gels 30 having an opaque boundary or divider 36 configured to reduce optical cross-talk between an optical source 18 and an optical detector 20 are illustrated. In effect, for both Orientation A and Orientation B, the opaque divider 36 separates the ear gel into an emitting section and a detecting section. In each embodiment, the ear gel 30 includes three sections 38a, 38b, 38c, as illustrated. Coupling between the skin and optical detector 20 greatly improves the chance that the optical detector 20 receives the desired biometric signal (AC component) and that coupling can be achieved with the use of the ear gel 30. However, optical cross-talk (light from the optical emitter 18 that enters the optical detector 20 but does not travel along a blood flow pathway and thus does not generate a signal that is modulated by blood flow) reduces the overall signal-to-noise detected by the optical detector. Compared with Section 38b and Section 38c, Section 38a has a reduced chance of making contact with the skin because its hemispherical shape is smaller than the auditory canal EC of an ear and part of the material is directed away from the ear canal EC (and towards the tympanic membrane).

Section 38b represents the portion of the ear gel 30 which is in optical communication with the optical source 18, through which light (unmodulated signal (DC component)) from the optical source 18 passes into the ear of a subject, and section 38c represents the portion of the ear gel 30 which is in optical communication with the optical detector 20, through which light (modulated signal (AC component) that is modulated by blood flow) from the ear returns to the optical detector 20. The desire is to create a sufficient, unobstructed amount of surface area in contact with the skin for the light to guide through while it moves into and out of the skin while minimizing cross talk between the optical source 18 and optical detector 20 that has not passed through the skin.

One approach to achieve this goal is presented in FIG. 9A. When the opaque divider 36 is oriented as shown in FIG. 9A, the divider 36 blocks crosstalk between the optical source 18 and optical detector 20, i.e., the unmodulated signal (DC component), but may also decrease the desired biometric signal (AC component). It is possible that this orientation prevents the light, shown as arrow A1, leaving the optical source 18 from being able to freely move through the circumferential geometry of the ear gel 30, across the entire 360° perimeter of the ear gel 30 and enter the skin of the subject. Thus, there is less chance for the light leaving the optical source 18 to enter the skin, cross the divider 36, and reenter the ear gel 30 on its path to the optical detector 20.

Stated another way, although the opaque divider 36 in this orientation helps prevent cross-talk between the optical source 18 and the detector 20, the light absorption also prevents circumferential light-guiding within the circular portion of the ear gel 30 (around the full circumference of Section 38b and Section 38c), thus reducing the opportunity for light from the optical source 18 to guide around the circumference of 38b and eventually exit into the skin (thereby modulate with blood flow) and also reducing the opportunity for the light beam modulated by blood flow to exit the skin and enter the circumference at Section 38c and guide within this region until it enters the optical detector 20.

To overcome this limitation, a configuration that may enable both high optical coupling and high optical cross-talk attenuation is presented in FIG. 9B. When the opaque divider 36 is oriented as shown in FIG. 9B, the divider 36 blocks optical crosstalk between the optical source 18 and the optical detector 20. However, the light beam, shown as the arrow A2, leaving the optical source 18 can freely traverse the circumferential perimeter of the ear gel 30, shown as Section 38b, until it enters radially into the skin and is modulated by blood flow (to create an AC PPG signal) and then reenters the ear gel 30 in Section 38C on its path to the optical detector 20.

Referring back to FIGS. 8A-8B in context of the aforementioned light-guiding, the cable 414 may be configured to provide light-guiding between the housing 410 and the sensor head assembly 10, 110, 210, 210′, 310. The light guiding materials and configurations may comprise the materials and configurations described above with reference to FIGS. 9A-9B and FIGS. 10A-10B (with light-guiding, cladding, a divider, etc.). A key benefit of this configuration is that the optical source 18 and optical detector 20 may be housed in the housing 410 rather than the sensor head assembly 10, 110, 210, 210′, 310. This may result in a reduction of size in the sensor head assembly 10, 110, 210, 210′, 310, thereby enabling the sensor head assembly 10, 110, 210, 210′, 310 to be more comfortable in the size-constrained ear canal.

A specific example, according to some embodiments of the present invention, is shown in FIG. 11. An opaque cable divider 1116 separates the sheath of the cable 414 into an emitting segment and a detecting segment, providing optical isolation between these segments and preventing optical cross-talk between them (as described above for FIGS. 9A-9B and FIGS. 10A-10B). The cable end 414a proximal to the housing 410 couples with at least one optical source 18 and at least one optical detector 20 supported by the housing 410. The cable distal end 414b couples with the ear gel 30, the ear gel 30 having its own circumferential divider 1136 to divide the ear gel 30 into emitting and detecting segments, such that the emitting segment of the cable 414 couples with the emitter segment of the ear gel 30 and the detecting segment of the cable 414 couples with the detecting segment of the ear gel 30.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1. A sensor head assembly, comprising:
a body member comprising an inlet port and an exit port at opposite ends thereof, and an internal passage extending between the inlet port and the exit port, wherein the body member is configured to be positioned within an ear canal of a subject;
an optical source secured to the body member at a first location; and
an optical detector secured to the body member at a second location different from the first location.

2. The sensor head assembly of claim 1, wherein the first and second locations are circumferentially spaced apart from each other.

3. The sensor head assembly of claim 1, wherein the first and second locations are adjacent each other.

4-5. (canceled)

6. The sensor head assembly of claim 1, further comprising an audio driver secured to the body member, wherein the audio driver is in acoustic communication with the internal passage of the body member.

7-8. (canceled)

9. The sensor head assembly of claim 1, further comprising:
an ear gel attached to the body member, wherein the ear gel comprises opposite inner and outer surfaces, wherein at least a portion of the ear gel is transparent to one or more selected wavelengths of light, and wherein the ear gel is configured to be inserted within the ear canal of the subject;
a first light guide having opposite first and second ends, wherein the first end of the first light guide is secured to the inner surface of the ear gel; and
a second light guide having opposite first and second ends, wherein the first end of the second light guide is secured to the inner surface of the ear gel;
wherein the second end of the first light guide is in optical communication with the optical source, wherein the second end of the second light guide is in optical communication with the optical detector, wherein the first light guide is configured to deliver light from the optical source into a first region of the ear canal, and wherein the second light guide is configured to collect light from a second region of the ear canal and deliver collected light to the optical detector.

10. The biometric monitoring device of claim 9, wherein the ear gel and the first and second light guides comprise a flexible optically transmissive material having a durometer measurement of between Shore A10 and Shore A80.

11. The biometric monitoring device of claim 9, wherein the ear gel comprises an opaque boundary or divider configured to reduce optical cross-talk between the optical source and the optical detector.

12. A sensor head assembly, comprising:
a body member comprising an inlet port and an exit port at opposite ends thereof, and an internal passage extending between the inlet port and the exit port, wherein the body member is configured to be positioned within an ear canal of a subject;
an audio driver secured to the body member, wherein the audio driver is in acoustic communication with the internal passage of the body member;
an annular member surrounding at least a portion of the audio driver;
an optical source secured to the annular member at a first location; and
an optical detector secured to the annular member at a second location different from the first location.

13. The sensor head assembly of claim 12, wherein the audio driver comprises a sound outlet tube, and wherein the sound outlet tube is secured within the internal passage of the body member via the inlet port.

14-17. (canceled)

18. The sensor head assembly of claim 12, wherein at least a portion of the audio driver, annular member, optical source and optical detector are encapsulated within a hydrophobic material.

19. The sensor head assembly of claim 12, further comprising:
an ear gel attached to the body member, wherein the ear gel comprises opposite inner and outer surfaces, wherein at least a portion of the ear gel is transparent to one or more selected wavelengths of light, and wherein the ear gel is configured to be inserted within the ear canal of the subject;
a first light guide having opposite first and second ends, wherein the first end of the first light guide is secured to the inner surface of the ear gel; and
a second light guide having opposite first and second ends, wherein the first end of the second light guide is secured to the inner surface of the ear gel;
wherein the second end of the first light guide is in optical communication with the optical source, wherein the second end of the second light guide is in optical communication with the optical detector, wherein the first light guide is configured to deliver light from the optical source into a first region of the ear canal, and wherein the second light guide is configured to collect light from a second region of the ear canal and deliver collected light to the optical detector.

20. (canceled)

21. The sensor head assembly of claim 19, wherein the ear gel comprises an opaque boundary or divider configured to reduce optical cross-talk between the optical source and the optical detector.

22. A sensor head assembly, comprising:
a body member comprising an inlet port and an exit port at opposite ends thereof, and an internal passage extending between the inlet port and the exit port, wherein the body member is configured to be positioned within an ear canal of a subject;
an audio driver secured to the body member, wherein the audio driver is in acoustic communication with the internal passage of the body member;
a flexible printed circuit secured to the audio driver;
an optical source secured to the annular member at a first location on the flexible printed circuit; and
an optical detector secured to the annular member at a second location on the flexible printed circuit different from the first location.

23. The sensor head assembly of claim 22, wherein the audio driver comprises a sound outlet tube, and wherein the sound outlet tube is secured within the internal passage of the body member via the inlet port.

24. The sensor head assembly of claim 22, wherein the first and second locations are circumferentially spaced apart from each other.

25. (canceled)

26. The sensor head assembly of claim 22, wherein the internal passage extends along a longitudinal centerline of the body member.

27. The sensor head assembly of claim 22, wherein at least a portion of the internal passage extends along a non-centerline portion of the body member.

28. (canceled)

29. The sensor head assembly of claim 22, further comprising:
an ear gel attached to the body member, wherein the ear gel comprises opposite inner and outer surfaces, wherein at least a portion of the ear gel is transparent to one or more selected wavelengths of light, and wherein the ear gel is configured to be inserted within the ear canal of the subject;
a first light guide having opposite first and second ends, wherein the first end of the first light guide is secured to the inner surface of the ear gel; and
a second light guide having opposite first and second ends, wherein the first end of the second light guide is secured to the inner surface of the ear gel;
wherein the second end of the first light guide is in optical communication with the optical source, wherein the second end of the second light guide is in optical communication with the optical detector, wherein the first light guide is configured to deliver light from the optical source into a first region of the ear canal, and wherein the second light guide is configured to collect light from a second region of the ear canal and deliver collected light to the optical detector.

30. The sensor head assembly of claim 29, wherein the ear gel and the first and second light guides comprise a flexible optically transmissive material having a durometer measurement of between Shore A10 and Shore A80.

31. The sensor head assembly of claim 29, wherein the ear gel comprises an opaque boundary or divider configured to reduce optical cross-talk between the optical source and the optical detector.

32-43. (canceled)
